(19) United States
(12) Patent Application Publication
(10) Pub. No.: US 2019/0350452 A1
(43) Pub. Date: Nov. 21, 2019

(54) VIRTUAL REALITY BASED VISUAL FIELD TESTING SYSTEM

(71) Applicant: Tracey Lynne Lewis, Summerfield, FL (US)

(72) Inventor: Tracey Lynne Lewis, Summerfield, FL (US)

(21) Appl. No.: 16/398,864

(22) Filed: Apr. 30, 2019

Related U.S. Application Data
(60) Provisional application No. 62/637,433, filed on Mar. 2, 2018.

(51) Int. Cl. A61B 3/024; A61B 3/18; A61B 3/00; G02B 27/01

(52) U.S. Cl. CPC A61B 3/024; A61B 3/18; G02B 27/017; A61B 3/0025; A61B 3/005

(57) ABSTRACT

A visual field-testing system is provided. The visual field-testing system include a method utilizing a virtual reality output device operatively associated with a central fixation standard/static automated perimetry visual field test. The virtual reality output device is embodied in goggles providing stationary high design technology enabling a fixed axial length from the lens of each eye being tested, producing highly accurate and repeatable testing results.

### CROSS-REFERENCE TO RELATED APPLICATION

This application claims the benefit of priority of U.S. provisional application No. 62/637,433, filed 2 Mar. 2018, the contents of which are herein incorporated by reference.

### BACKGROUND OF THE INVENTION

The present invention relates to visual field-testing systems and methods and, more particularly, a visual field-testing system incorporating a virtual reality output device.

Visual field testing is an extremely important tool for diagnosis of glaucoma, stroke, macular degeneration, multiple sclerosis, drug toxicity, etc. The assessment of peripheral vision in ophthalmology currently uses a large bulky visual field machine—the gold standard machine—called a “Humphrey.” Made by Zeiss.

The Humphrey visual field test requires patients to be mobile so as to access the Humphrey equipment in a room dedicated to housing the Humphrey visual field machinery. The patient also needs to be responsive and alert in order to maintain a good test; for instance, head position is so important that if the patient backs up or moves forward in the machine, the test is invalid. Similarly, if the patient looks away, the test is invalid. Each eye takes over twenty minutes, which is a long time for patients to hold still. As a result, there is no way to test the visual field on a bed bound patient, for example in a nursing home or an elderly, disabled patient.

In the context of diagnosing strokes, the sooner a physician can administer TPA to reverse cell death the better—TPA is most effective, though, only when used within 6 hours of stroke. Currently a CT scan is the gold standard of stroke-diagnostic tools. In the emergency room, however, it can take about 24 hours to diagnose a stroke because the anoxia—i.e., the swelling and subsequent death of brain cells—may not show up on MRI or CT scan for approximately one day. During this time period, TPA is not used because the diagnosis is not a certainty, and thus risk may not out way benefit—to the detriment of the patient suffering from an undiagnosed stroke.

Notably, there are certain visual strokes that can be diagnosed if a visual field test could be accurately obtained. It would then be possible to administer TPA and reverse the devastation. However, because a patient suffering from a stroke may not be able to properly perform a visual field test (because of the aforementioned requirements) they may go untreated.

As can be seen, there is a need for a visual field testing system incorporating a virtual reality output device adapted to represent the visual aspects of the visual field test, whereby the output device is embodiment in goggles adapted with stationary high design technology establishing a fixed axial length from the face of the wearer, enabling fixed central point operation during testing—in other words, the output device moves with the patient's head—an embedded foveal camera can track fixation, this would result in greater reproducibility of the visual field test through ameliorating the head and eye fixation related problems. Thus, incorporation of the virtual reality output device facilitates testing of bed-bound patients, it also makes the system portable and available to all patients including gurney-bound patients.

### SUMMARY OF THE INVENTION

In one aspect of the present invention, a method of providing visual field-testing, includes the following: providing a virtual reality output device configured to represent the visual aspects of a visual field test through a user interface of said virtual reality output device, wherein the virtual reality output device maintains a fixed axial distance between the user interface and an eye lens of each eye being tested; a foveal detection monitor, the virtual reality output device adapted to be wearable on a head of a human user; and operatively associating systemic software with the virtual reality output device, wherein the systemic software is configured to provide central fixation standard/static automated perimetry testing through the user interface, wherein the virtual reality output device are goggles adapted to be worn over the eyes of a human user, wherein the central fixation standard/static automated perimetry testing includes selectively representing discrete targets flashed on the user interface at high medium and low intensity so that fixed point and perimetry adjustments measure from 10 degrees up to 135 degrees of a visual field of the human user, and wherein the goggles provide stationary structure for facilitating the fixed axial distance.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a schematic view of an exemplary embodiment of a visual field test of the present invention, illustrating a view of a visual field test as seen through a virtual reality output device 10;

FIG. 3 is a perspective view of an exemplary embodiment of the present invention operatively associated with a computer 16; and

FIG. 4 is a perspective view of an exemplary embodiment of the present invention operatively associated with a smart device 18.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a visual field-testing system incorporating a virtual reality output device operatively associated with a central fixation standard/static automated perimetry visual field test. The virtual reality output device is embodied in goggles providing stationary high design technology enabling a fixed axial length from the lens of each eye being tested, producing highly accurate and repeatable testing results.

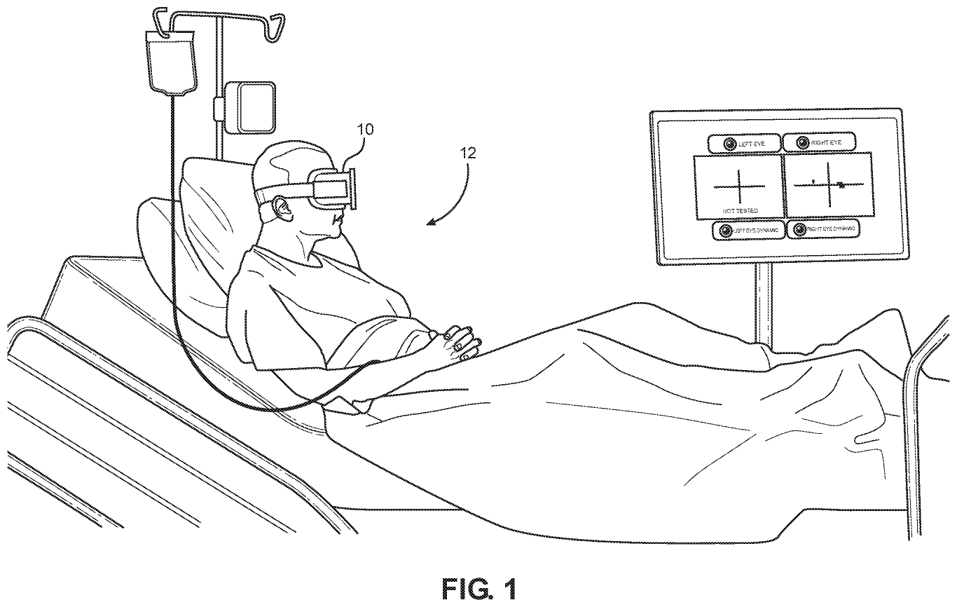

Referring now to FIGS. 1 through 4 the present invention may include at least one computer device 16 or 18 with a user interface. The computing device 16 or 18 may include at least one processing unit and a form of memory including, but not limited to, a desktop, laptop, and smart device, such as, a tablet and smart phone computer. The computing device 16 or 18 may include a program product including a machine-readable program code for causing, when executed, the computer to perform steps. The program product may include software which may either be loaded onto the computing device 16 or 18 or accessed by the computing device. The loaded software may include an application on a smart device. The software may be accessed by the computing device 16 or 18 using a web browser. The computing device may access the software via the web browser using the internet, extranet, intranet, host server, internet cloud and the like.

The virtual reality output device 10 may embody goggles adapted to be worn over the eyes of a user 12. The virtual reality output device 10 may be configured to utilize advanced technology 180-degree virtual reality goggles that are operatively associated with a central fixation standard/static automated perimetry visual field test 14. The goggles of the virtual reality output device 10 are a stationary high design technology with a fixed axial length from the face. Therefore, said goggles cannot be adjusted forward or backward or away even with head turning or misplacement of the goggle. This allows for a fixed central point and highly accurate testing. One eye is patched while the test is run on the other eye.

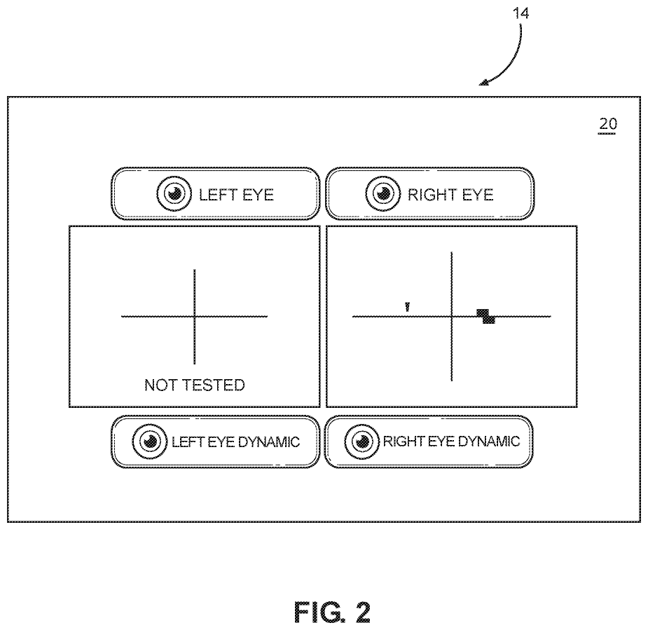

The central fixation standard/static automated perimetry visual field test 14 may be driven by the abovementioned software application configured to provide electronically represented automated visual fields through the user interface 20 of the virtual reality output device 10, as illustrated in FIG. 2.

The computing device 16 or 18 may be electrically coupled to the virtual reality output device 10 so that the systemic software (loaded on the computing device 16 or 18) may provide the test and analyze the test's resulting data sets, including progression analyses over time. The software application provides a standard/static automated perimetry test, wherein each eye is monitored separately using an eye patch on the non-tested eye, as illustrated in FIG. 2. Discrete targets are flashed at high medium and low intensity wherein a fixed point and perimetry adjustments can be made to measure from 10 degrees up to 130 degrees of visual field. The results are subjective to what the patient sees and the patient uses a wired or wireless clicker, pressing the button each time a light is flashed. Red dot testing and Amsler grids can also be obtained in a similar fashion.

The results are exportable to a printer or the cloud, via the computing device 16 or 18. A decibel format and/or grayscale format can be computed for both generalized and pattern depression and either printed or scanned to a HIPPA compliant patient chart. The present invention's method of visual field testing can monitor presence or absence of glaucoma, progression of glaucoma, cerebral vascular accident in the occipital cortex or visual pathways, worsening or flare of multiple sclerosis, macular degeneration and other visually disabling diseases in both bed-bound and ambulatory patients.

A similar but less technologically advanced form of testing can be obtained via a wireless virtual reality output device 10 (virtual reality goggles) and a smart phone/computing device 18, enabling patients to test themselves in the privacy of their own homes. The clicker may be Bluetooth or equivalently wireless so as not to need to be physically connected. The results can be used for initial screening or monitoring of progression of eye disease and again, due to axial length fixation of the goggles, the results are repeatable.

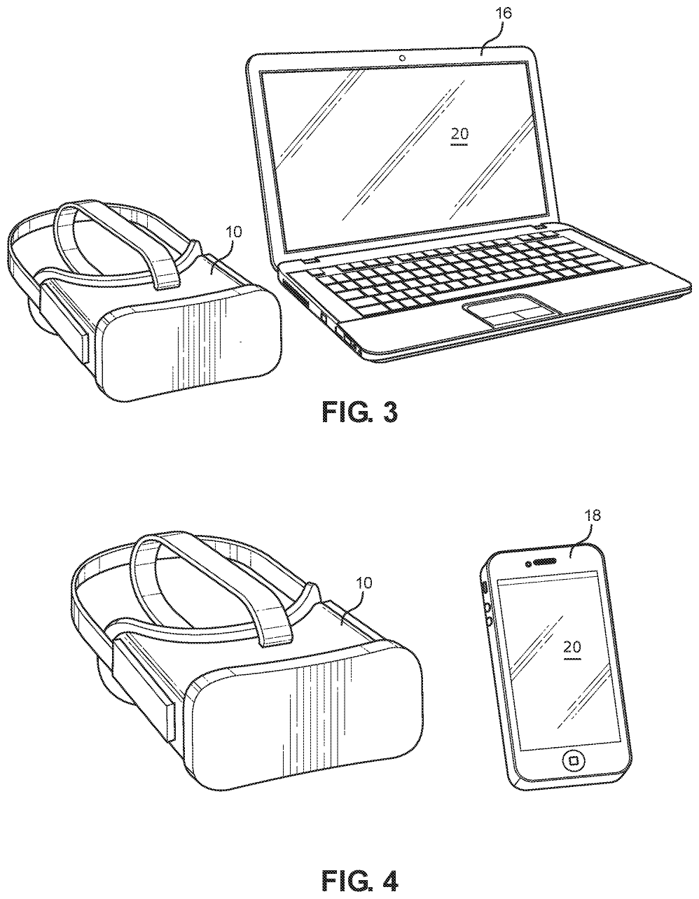

Referring to FIGS. 3 and 4, the present invention includes at least a first embodiment (FIG. 3) and a second embodiment (FIG. 4). The first embodiment differs from the second embodiment in the following ways: two different headsets. The first embodiment includes a high design—like an Oculus Rift—to interface with the laptop computing device 16. The second embodiment to be freestanding, inexpensive, to interface with a PDA or smart phone computing device 18. In either case, using the virtual reality goggles 10, the results are similar or better to standardized Zeiss owned Humphrey VF testing equipment.

From the collected data, the data sets may be compiled in HIPPA compliant fashion, and the results could also be sent to the physician as a baseline and reviewed with the patient upon each patient visit to the office as recommended.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A method of providing visual field-testing, comprising:
providing a virtual reality output device configured to represent the visual aspects of a visual field test through a user interface of said virtual reality output device, wherein the virtual reality output device maintains a fixed axial distance between the user interface and an eye lens of each eye being tested; one eye patched while second eye is being tested
the virtual reality output device adapted to be wearable on a head of a human user; with integrated foveal fixation device and
operatively associating systemic software with the virtual reality output device, wherein the systemic software is configured to provide central fixation standard/static automated perimetry testing through the user interface.

2. The method of claim 1, wherein the virtual reality output device are goggles adapted to be worn over the eyes of a human user.

3. The method of claim 1, wherein the central fixation standard/static automated perimetry testing includes selectively representing discrete targets flashed on the user interface at high medium and low intensity so that fixed point and perimetry adjustments measure from 10 degrees up to 135 degrees of a visual field of the human user.

4. The method of claim 2, wherein the goggles provide stationary structure for facilitating the fixed axial distance.
